(19) United States
(12) Patent Application Publication
(10) Pub. No.: US 2009/0110698 A1
(43) Pub. Date: Apr. 30, 2009

(54) METHOD OF DETERMINING VACCINE COMPLIANCE

(75) Inventors: Benjamin M. Hause, Currie, MN (US); Randy R. Simonson, Worthington, MN (US); Russell F. Bey, Arden Hills, MN (US); Douglas L. Stine, Overland Park, KS (US)

Correspondence Address: BRIGGS AND MORGAN P.A., 2200 IDS CENTER, 80 SOUTH 8TH ST, MINNEAPOLIS, MN, 55402, US

(73) Assignee: Newport Laboratories, Inc., Worthington, MN (US)

(21) Appl. No.: 11/931,328

(22) Filed: Oct. 31, 2007

(51) Int. Cl. A61K 39/00; A61P 43/00; C12N 9/24

(52) U.S. Cl. 424/206.1; 424/184.1; 435/200

(57) ABSTRACT

A protein (xylanase) has been identified that produces a strong immune response in pigs when added to a vaccine. The protein is added to vaccines at a particular concentration. The protein is delivered to the animal as part of the vaccine and elicits an immune reaction (antibodies are generated by the animal against the compliance marker protein). The antibodies are then detected in sera samples by a diagnostic test, enzyme-linked immunosorbent assay (ELISA). In the ELISA compliance marker assay, the marker protein (antigen) in coated onto polystyrene plates and is used to detect antibodies against the marker in sera samples. This combination of marker protein added to vaccines and the ELISA to detect antibodies raised against the marker protein can be used to determine whether a particular animal(s) has been vaccinated. The selected protein is commercially-available, identified as Generally Recognized as Safe by the FDA, and does not cause adverse reactions in animals.

### BACKGROUND OF THE INVENTION

A large problem in the food livestock industry is the proper vaccination of animals. Many food animals are raised in high density confinement settings where vaccination is an essential tool to manage herd health. Increasingly, large corporations own livestock and contract out the rearing of the animals. When disease breaks occur in these systems in animals that were supposedly vaccinated, questions arise as to whether the vaccine failed to protect the animals or whether the vaccine was delivered properly. The latter possibility regarding proper delivery of the vaccine is referred to as vaccine compliance.

Due to questions regarding vaccine compliance, producers desire a method to determine whether an animal has been vaccinated. As vaccines work by stimulating the host's immune system to generate antibodies against a particular antigen, it is nearly impossible to differentiate antibodies resulting from vaccination from antibodies formed in response to natural infection. An alternate method is thus required to differentiate antibodies arising from vaccination such that natural infection can be eliminated.

The present invention is a compliance marker. A particular biological molecule is added to vaccines at a particular concentration. The biological molecule is delivered to the animal as part of the vaccine and elicits an immune reaction (antibodies are generated by the animal against the compliance marker protein). The antibodies are then detected in sera samples by a diagnostic test, enzyme-linked immunosorbent assay (ELISA). In the ELISA compliance marker assay, the marker (antigen) is coated onto polystyrene plates and is used to detect antibodies against the marker in sera samples. This combination of marker added to vaccines and the ELISA to detect antibodies raised against the marker can be used to determine whether a particular animal(s) has been vaccinated.

Several factors are critical in selecting a suitable compliance marker. The marker needs to be safe as it is being introduced into the human food supply. Proteins categorized as “General Recognized As Safe (GRAS)” by the Food and Drug Agency (FDA) are suitable. Additionally, the marker needs to be immunogenic as a strong immune response is needed for sensitive determination of vaccination status. The marker needs to be readily available and economical to add to vaccines. Also, animals should not be naturally exposed to significant levels of the marker such that false positives are problematic. It is also critical that the marker does not cause any adverse reactions in the animal.

An initial marker that meets all the requirements laid out in the previous paragraph is xylanase originating from Thermomyces lanuginosus. This protein is commercially available. It is produced by Novozymes and is produced under the trade name “Pentopan Mono BG”. The xylanase protein from Thermomyces lanuginosus is expressed recombinantly in Aspergillus oryzae by submerged fermentation. The xylanase is used commercially in the baking industry as it helps condition dough. Xylanase has also been used in the poultry industry as a feed additive as it helps improve daily weight gain. Xylanase has received GRAS status and is produced at a large scale and is readily available at a cost that makes incorporation into vaccines economically feasible. Inclusion of xylanase into vaccine preparations resulted in a strong immune response as detected by a xylanase-based ELISA. All of the previously mentioned factors point to the suitability of xylanase as a vaccine compliance marker.

### SUMMARY OF THE INVENTION

The present invention is a compliance marker. A particular biological molecule is added to vaccines at a particular concentration. The biological marker is delivered to the animal as part of the vaccine and elicits an immune reaction (antibodies are generated by the animal against the compliance marker). The antibodies are then detected in sera samples by a diagnostic test, enzyme-linked immunosorbent assay (ELISA). In the ELISA compliance marker assay, the marker (antigen) is coated onto polystyrene plates and is used to detect antibodies against the marker in sera samples. This combination of marker added to vaccines and the ELISA to detect antibodies raised against the marker can be used to determine whether a particular animal(s) has been vaccinated.

A principal object and advantage of the invention is that it provides a marker which can be used as a compliance marker in vaccines to distinguish vaccinated from unvaccinated animals.

Another principal object and advantage of the present invention is that it uses a commercially-available protein as a compliance marker.

Another principal object and advantage of the present invention is that the commercial protein used as a compliance marker is categorized by the FDA as Generally Recognized as Safe (GRAS).

Another principal object and advantage of the present invention is that the protein selected as a compliance marker produces a strong immune response in animals.

Another principal object and advantage of the present invention is that the protein selected as a compliance marker does not cause adverse reactions in the animal.

Another principal object and advantage of the present invention is that animals are not naturally exposed to significant levels of the protein selected as the compliance marker.

### BRIEF DESCRIPTION OF DRAWINGS

FIG. 1 is a bar graph showing the results of an ELISA assay of three candidate marker proteins.

FIG. 2 is a line graph showing the results of an ELISA assay for xylanase as a marker protein in nursery pigs.

FIG. 3 is a line graph showing the results of an ELISA assay for xylanase as a marker protein in finisher pigs.

FIG. 4 is a line graph showing the results of an ELISA assay for xylanase in nursery age pigs using Trigen as an adjuvant.

FIG. 5 is a line graph showing the results of an ELISA assay for xylanase in nursery age pigs using aluminum hydroxide as an adjuvant.

FIG. 6 is a bar graph showing the results of an ELISA assay for pregnant sows.

### DETAILED DESCRIPTION OF THE PREFERRED EMBODIMENT

Xylanase (catalog number X2753) was purchased from Sigma Aldrich (St. Louis, Mo.). The xylanase was produced by Novozymes Corporation under the trade name “Pentopan Mono BG”. The xylanase gene originated from the fungus Thermomyces lanuginosus and the xylanase protein was produced recombinantly in a genetically modified Aspergillus oryzae by submerged fermentation. The xylanase purified from fermentation and has endo 1,4-β-xyalanse activity.

Xylanase was added to vaccine preparations from a sterile stock solution to a final concentration of 1 mg/mL. To make the sterile stock solution of xylanase, a 100 mg/ml solution of xylanase in phosphate buffered saline (PBS) was prepared and formalin was added to a final concentration of 0.4%. The stock solution was incubated 18-24 hours at 37° C. before use. Stock solution was added at a rate of 1% to vaccines such that the final concentration of sterile marker protein in the vaccine was 1 mg/mL.

### Marker Protein ELISA

A 1 mg/mL solution of marker protein was made in 0.1 M carbonate buffer solution, pH 9.6. 100 μL of marker protein solution was added to all wells of a 96-well polystyrene microtiter plate (Immulon 2HB). Plates were incubated 24 hours at 4° C. Plates were washed four times with wash buffer (phosphate buffered saline+0.05% Tween 20). Plates were blocked with 200 μL/well of SuperBlock Reagent (Pierce Biotechnology). Plates were washed four times with wash buffer. Sera samples were diluted 1:400 in sample dilution buffer (SuperBlock Reagent+0.05% Tween 20). 100 μL of diluted sample was added to microtiter wells in duplicate and incubated for 30 minutes at 37° C. Plates were washed four times with wash buffer. Goat anti-pig IgG peroxidase conjugate was diluted 1:2000 in sample dilution buffer. 100 μL of diluted antibody was added to each well and incubated 30 minutes at 37° C. Plates were washed four times. 100 μL of OPD solution (Sigma Aldrich P9187) was added to each well and incubated at 37° C. for 15 minutes. The color development reaction was stopped by the addition of 1100 μL of 0.1N HCl. Absorbance was read for each well at 495 mm.

### Example 1

### Screening for Candidate Compliance Marker Proteins

Three proteins (cellulase, xylanase and amylase) were screened for use as compliance markers as they met several of the criteria previously described (commercially available, low-cost, GRAS). Proteins were added to a Newport Laboratories swine influenza virus vaccine to a final concentration of 1 mg/mL. 2 mL of vaccine was delivered intra-muscularly to each pig on day 0 and day 14. Pigs were bled at day 28 and the sera were analyzed by ELISA. Fifteen pigs received the vaccine without any marker proteins. Five pigs received each marker protein.

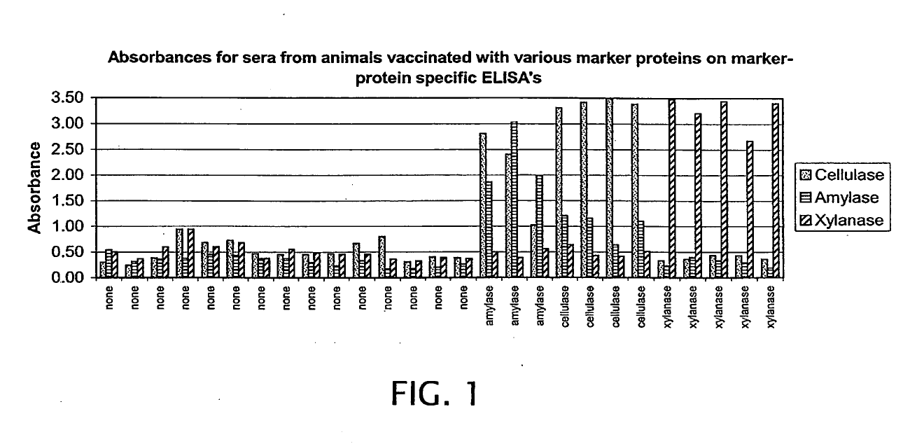

Sera collected at day 28 was analyzed by ELISA assays designed to detect the three marker proteins. ELISA plates were generated by coating a 1 mg/mL solution of each marker protein and run according to the protocol above. Results are shown in Table 1 and FIG. 1. All three marker proteins elicited an immune response that was detectable on the corresponding ELISA. Antibodies against cellulase and amylase were cross-reactive with each other, limiting their usefulness. Additionally, two pigs receiving amylase and 1 pig receiving cellulase died during the course of experiment (post mortem examinations were not performed to determine cause of death). All five pigs vaccinated with xylanase seroconverted to a high extent. No cross-reactivity was observed with the xylanase-vaccinated pigs and the other marker protein ELISA's and non-vaccinated pigs had low background titers on the xylanase ELISA. Pigs receiving xylanase showed no adverse reactions.

Results of this initial screening study show that xylanase appears suitable for use as a vaccine compliance marker.

TABLE 1
Absorbances for sera collected from pigs vaccinated with various
marker proteins on marker-protein specific ELISA's

| Marker | | | | |
|---|---|---|---|---|
| Protein | | cellulase | amylase | xylanase |
| in Vaccine | Pig # | Absorbance | absorbance | absorbance |
| none | 680 | 0.30 | 0.54 | 0.50 |
| none | 666 | 0.24 | 0.31 | 0.37 |
| none | 681 | 0.38 | 0.36 | 0.59 |
| none | 669 | 0.95 | 0.38 | 0.94 |
| none | 695 | 0.68 | 0.45 | 0.60 |
| none | 653 | 0.73 | 0.44 | 0.69 |
| none | 657 | 0.47 | 0.37 | 0.38 |
| none | 652 | 0.45 | 0.37 | 0.56 |
| none | 661 | 0.45 | 0.30 | 0.48 |
| none | 698 | 0.47 | 0.23 | 0.45 |
| none | 649 | 0.67 | 0.33 | 0.46 |
| none | 683 | 0.80 | 0.17 | 0.36 |
| none | 686 | 0.31 | 0.18 | 0.32 |
| none | 670 | 0.40 | 0.19 | 0.38 |
| none | 694 | 0.38 | 0.26 | 0.37 |
| amylase | 699 | 2.81 | 1.87 | 0.50 |
| amylase | 671 | 2.40 | 3.03 | 0.38 |
| amylase | 692 | 1.02 | 1.99 | 0.56 |
| cellulase | 663 | 3.31 | 1.21 | 0.64 |
| cellulase | 646 | 3.41 | 1.16 | 0.43 |
| cellulase | 656 | 3.49 | 0.64 | 0.42 |
| cellulase | 700 | 3.39 | 1.10 | 0.51 |
| xylanase | 647 | 0.33 | 0.23 | 3.47 |
| xylanase | 672 | 0.36 | 0.39 | 3.21 |
| xylanase | 664 | 0.43 | 0.33 | 3.44 |
| xylanase | 659 | 0.43 | 0.30 | 2.67 |
| xylanase | 648 | 0.36 | 0.19 | 3.40 |

### Example 2

### Evaluation of the Xylanase Marker Protein in Different Age Pigs

A 1 mg/mL solution of xylanase in phosphate buffered saline was prepared using Newport Laboratories' proprietary oil and water emulsion adjuvant “Trigen” added to 10%. A 2 cc dose was given to ten nursery age pigs (˜3 weeks old) and ten finisher age pigs (˜12 weeks old). A blood sample was collected from each pig prior to being vaccinated. A follow up booster injection was given two weeks later (day 14) and another blood sample was collected. Blood collection continued every two weeks for approximately two months. Sera samples were run on the xylanase ELISA as previously described. An S/P ratio greater than 0.2 represents positive signal for xylanase antibodies.

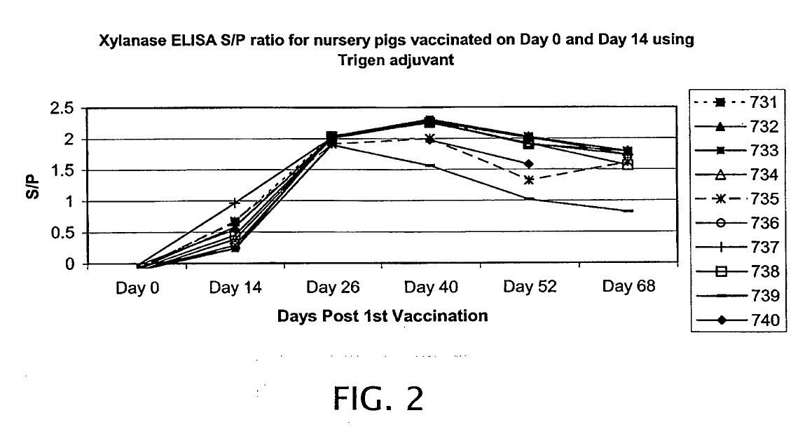

Nursery pigs were all negative on the xylanase ELISA on day 0. Following a single vaccination on day 0, all pigs seroconverted on the ELISA by day 14 and remained positive for the duration of the experiment (FIG. 2).

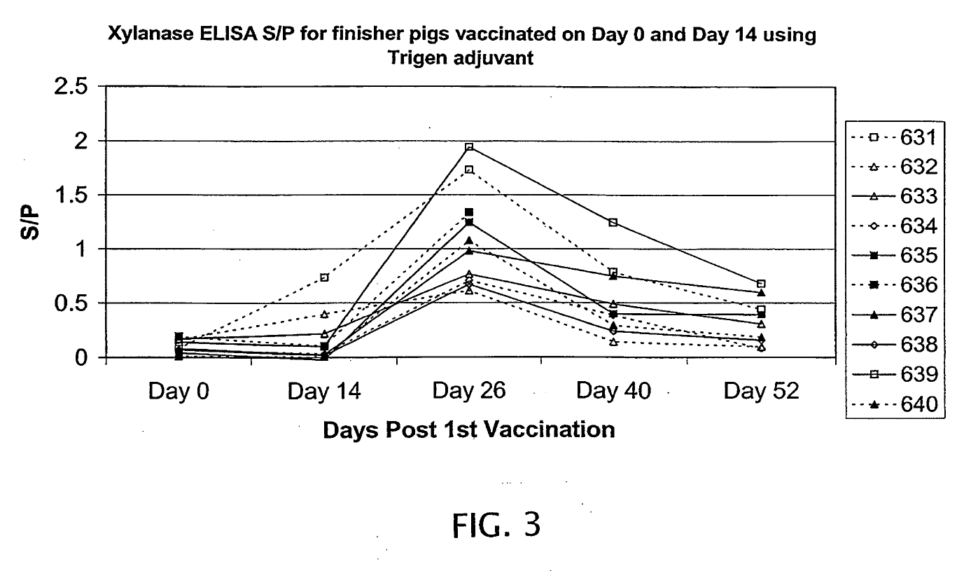

Finisher pigs were all negative on the xylanase ELISA on day 0. Following a single vaccination on day 0, 30% of the pigs seroconverted on the ELISA. By day 26, all ten animals were positive for xylanase and a majority of them remained positive through the duration of the experiment. (FIG. 3)

Results from this experiment show that xylanase is suitable as a compliance marker in both nursery and finisher age pigs.

### Example 3

### Evaluation of the Xylanase Marker Protein Using Two Different Adjuvants

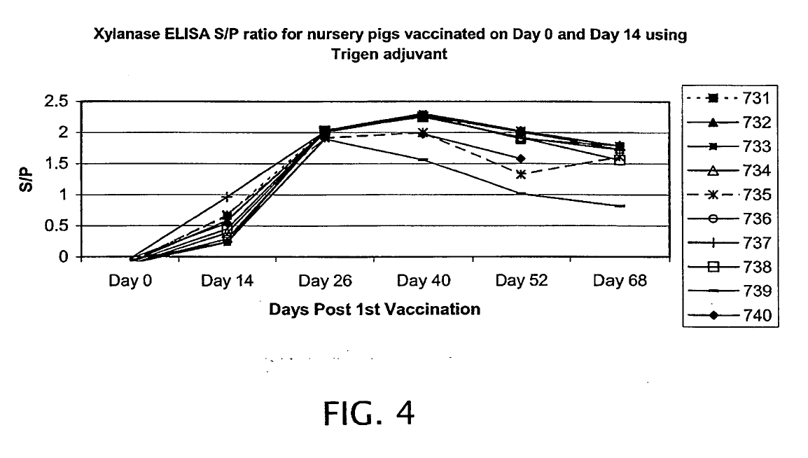

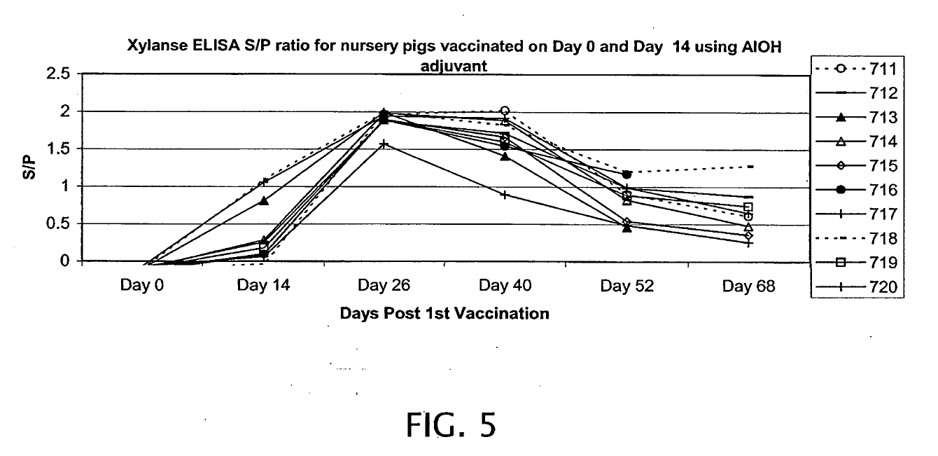

A 1 mg/mL solution of xylanase in phosphate buffered saline was prepared using Newport Laboratories' proprietary oil and water emulsion adjuvant “Trigen” added to 10% or the commonly used adjuvant aluminum hydroxide. A 2 cc dose containing Trigen adjuvant (FIG. 4) or AlOH adjuvant (FIG. 5) was given to ten nursery age pigs (˜3 weeks old) each. A blood sample was collected from each pig prior to being vaccinated. A follow up booster injection was given two weeks later (day 14) and another blood sample was collected. Blood collection continued every two weeks for approximately two months. Sera samples were run on the xylanase ELISA as previously described. An S/P ratio greater than 0.2 represents positive signal for xylanase antibodies.

Nursery pigs were all negative on the xylanase ELISA on day 0. Following a single vaccination using Trigen adjuvant on day 0, all pigs seroconverted on the ELISA by day 14 and remained positive for the duration of the experiment (FIG. 4).

Nursery pigs were all negative on the xylanase ELISA on day 0. Following a single vaccination using AlOH adjuvant on day 0, 50% pigs seroconverted on the ELISA by day 14 (FIG. 5). Following a booster dose on day 14, all pigs were seropositive for xylanase antibodies on day 26 and remained that way throughout the duration of the experiment.

Results from this experiment show that xylanase is a suitable compliance marker when used with either Trigen or aluminum hydroxide adjuvants.

### Example 4

### Evaluation of the Xylanase Marker Protein in Sows and Maternal Antibody Transfer

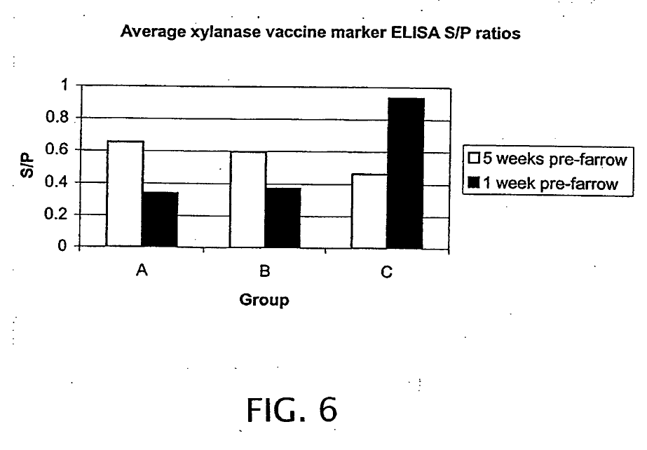

A 1 mg/mL solution of xylanase in phosphate buffered saline was prepared using Newport Laboratories' proprietary oil and water emulsion adjuvant “Trigen” added to 10%. A 2 cc dose was given to sows at 5 weeks prior to farrowing and blood was collected. A second 2 cc dose was given 2 weeks later (3 weeks pre-farrow) and blood was again collected. A final bleed was performed 1 week prior to farrowing. Seventeen sows were not-vaccinated and served as controls (Group A). Eighteen sows were vaccinated only once at 5 weeks pre-farrowing (Group B). Seventeen sows were vaccinated at both five and three weeks pre-farrowing (Group C). Sera were run on the marker protein ELISA to detect antibodies for xylanase. Group average ELISA S/P values are shown in FIG. 6. Only Group C (sows given two doses of xylanase marker protein) seroconverted to the marker.

Following farrowing, blood was collected from five piglets from each sow in groups A-C when the piglets reached 3 weeks of age. Sera were run on the xylanase marker ELISA. All groups had negative average ELISA titers.

The results from this experiment demonstrate that the xylanase compliance marker is suitable for use in sows and that maternal antibodies for xylanase do not appear to transfer to piglets. This latter point is desirable as it allows for use of the vaccine marker in piglets born from sows that had been exposed to the marker.

Unless otherwise defined, all technical and scientific terms used herein have the same meaning as commonly understood by one of ordinary skill in the art to which this invention belongs. Although methods and materials similar to or equivalent to SF those described herein can be used in the practice or testing of the present invention, suitable methods and materials are described below. All publications, patent applications, patents, and other references mentioned herein are incorporated by reference in their entirety to the extent allowed by applicable law and regulations. In case of conflict, the present specification, including definitions, will control.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

What is claimed:

1. A composition of matter comprising a naturally-occurring protein in combination with a vaccine, the naturally-occurring protein further comprising purified xylanase.

2. The composition of matter of claim 1, wherein the purified xylanase is produced from the xylanase gene of Thermomyces lanuginosus.

3. The composition of matter of claim 1, wherein the xylanase produces a detectable immune response in an injected animal, the detectable immune response being differentiated from an immune response to the vaccine.

4. The composition of matter of claim 1, wherein the vaccine is a swine influenza virus vaccine.

5. A method for assaying the presence of a marker protein in a vaccine injected into an animal, comprising the steps of:
(a) adding the marker protein to a vaccine;
(b) injecting the vaccine and marker protein into the animal;
(c) collecting sera from the injected animal; and
(d) analyzing the collected sera for antibodies to the marker protein.

6. The method of claim 5, wherein the step of analyzing the sera for antibodies to the marker protein is carried out using the ELISA protocol.

7. The method of claim 6, wherein the marker protein further comprises xylanase.

8. The method of claim 6, wherein the vaccine is a swine influenza virus vaccine.

9. A naturally-occurring protein for testing the proper delivery of a vaccine to an animal, the naturally-occurring protein producing a detectable immune response when injected into an animal simultaneously with a vaccine.

10. The naturally-occurring protein of claim 9, wherein the naturally-occurring protein comprises xylanase.

11. The naturally-occurring protein of claim 9, wherein the naturally-occurring protein is commercially-available.

12. The naturally-occurring protein of claim 9, wherein the naturally-occurring protein is recognized as safe by the Food and Drug Administration.

13. The naturally-occurring protein of claim 9, wherein the vaccine is a swine influenza virus vaccine.

14. A method of screening naturally-occurring biological molecules for use as a vaccine-compliance marker, comprising the steps of:
(a) adding each of the biological modules to be screened to a solution of the vaccine;
(b) injecting the solution of biological molecule and vaccine into an animal;
(c) obtaining a sample of blood from the animal;
(d) measuring an immune response induced by each biological molecule;
(e) measuring a cross-reactive immune response for each biological molecule against each of the other biological molecules; and
(f) selecting as a vaccine-compliance marker the biological molecule that has an immune response in step (d) and the smallest cross-reactive immune response against each of the other biological molecules in step (e).

15. The method of claim 12, wherein steps (d) and (e) are performed using an ELISA assay.

16. The method of claim 12, wherein the animal is a pig.

17. The method of claim 14, wherein the vaccine is a swine influenza virus vaccine.

18. The method of claim 12, wherein the biological molecules are proteins.
